(19) United States
(12) Patent Application Publication
(10) Pub. No.: US 2021/0244385 A1
(43) Pub. Date: Aug. 12, 2021

(54) ULTRASOUND DIAGNOSTIC APPARATUS, ULTRASOUND PROBE SWITCHING METHOD, AND STORAGE MEDIUM

(71) Applicant: Konica Minolta, Inc., Tokyo (JP)

(72) Inventor: Takashi SAKAI, Yokohama-shi, Kanagawa (JP)

(21) Appl. No.: 17/154,415

(22) Filed: Jan. 21, 2021

(30) Foreign Application Priority Data
Feb. 7, 2020 (JP) 2020-019432

(51) Int. Cl. A61B 8/00; A61B 8/08

(52) U.S. Cl. CPC A61B 8/4472; A61B 8/5207; A61B 8/565; A61B 8/4438; A61B 8/4405; A61B 8/4245

(57) ABSTRACT

An ultrasound diagnostic apparatus to which a plurality of ultrasound probes are connected and which transmits and receives ultrasound using one of the plurality of ultrasound probes to generate ultrasound image data, the ultrasound diagnostic apparatus includes the following, a detector which optically detects the ultrasound probe which is used among the plurality of ultrasound probes; and a hardware processor which switches and sets the detected ultrasound probe to be the ultrasound probe which is used.

### CROSS-REFERENCE TO RELATED APPLICATIONS

The entire disclosure of Japanese Patent Application No 2020-019432 filed on Feb. 7, 2020 is incorporated herein by reference in its entirety.

### BACKGROUND

### Technological Field

The present invention relates to an ultrasound diagnostic apparatus, an ultrasound probe switching method, and a storage medium.

### Description of the Related Art

By performing ultrasound diagnosis, a state of a heart or a fetus can be understood from an ultrasound image by a simple operation such as placing an ultrasound probe against a surface of the body of the subject or placing the ultrasound probe inside the body of the subject. Further, since the ultrasound diagnosis is highly safe, the examination can be repeatedly performed. There is a well-known ultrasound diagnostic apparatus used for performing ultrasound diagnosis.

There is a well-known ultrasound diagnostic apparatus in which a plurality of ultrasound probes are connected to an ultrasound image diagnostic apparatus main body, and one ultrasound probe is selected from the plurality of ultrasound probes and used. Conventionally, when the ultrasound probe to be used is selected, the person who performs the examination such as the doctor or the examination technician needed to operate a button on an operation inputter of the ultrasound image diagnostic apparatus main body or a button to be touched displayed on the display. When the ultrasound probe is already selected, it is necessary to view the screen and determine whether a switching operation is necessary to omit the switching operation.

There is a well-known ultrasound diagnostic apparatus in which the switching of the ultrasound probe is made easier (JP 2000-107176). For example, a sensor is provided in the ultrasound probe, and a main body side sensor is provided in the probe holder. The main body side sensor detects the sensor on the probe, and the ultrasound probe not held in the probe holder is detected. With this, the ultrasound probe to be used is switched.

There is a well-known ultrasound diagnostic apparatus in which a movement detection unit is provided in the ultrasound probe and when the movement of the ultrasound probe is detected, control to switch the ultrasound probe is performed (JP 2015-134031). There is a well-known ultrasound diagnostic system in which a sensor which detects touch is attached to the ultrasound probe, and when the sensor detects that the person who performs the examination is holding the ultrasound probe, the ultrasound probe to be used is switched (JP 2006-187589).

There is a well-known ultrasound medical diagnostic apparatus in which a capacitance type or mechanical type selection switch is attached to the ultrasound probe, the ultrasound medical diagnostic apparatus detects that the person who performs the examination holds the ultrasound probe with the hand, and the ultrasound probe to be used is switched (JP 2000-14670). Alternatively, there is a well-known ultrasound diagnostic apparatus in which a touch sensor which detects touch is attached to the ultrasound probe (JP H5-245140). When the sensor detects that the person who performs the examination holds the ultrasound probe with the hand, the ultrasound probe to be used is switched.

### SUMMARY

However, according to the ultrasound image diagnostic apparatus described in JP 2000-107176, the relation between the positions of the ultrasound probe and the probe holder need to be maintained in the same state. In actual use, the combination of the ultrasound probe to be used changes for each patient and the ultrasound probe with the high possibility of use is set in advance in a position near the person who performs the examination Therefore, it is difficult to maintain the relation of the positions between the ultrasound probe and the probe holder.

Turning to the apparatuses described in JP 2015-134031, JP 2006-187589, JP 2000-14670, and JP H5-245140, it is difficult to adjust the existing ultrasound probes to these apparatuses, and if the new ultrasound probes include a sensor or a switch, the configuration becomes complicated leading to increase in costs.

The present invention is conceived in view of the above problems, and the purpose of the present invention is to easily switch the ultrasound probe to be used using existing ultrasound probes.

To achieve at least one of the above-mentioned objects, according to an aspect of the present invention, an ultrasound diagnostic apparatus reflecting one aspect of the present invention is an ultrasound diagnostic apparatus to which a plurality of ultrasound probes are connected and which transmits and receives ultrasound using one of the plurality of ultrasound probes to generate ultrasound image data, the ultrasound diagnostic apparatus including: a detector which optically detects the ultrasound probe which is used among the plurality of ultrasound probes; and a hardware processor which switches and sets the detected ultrasound probe to be the ultrasound probe which is used.

According to another aspect, an ultrasound diagnostic apparatus to which a plurality of ultrasound probes are connected and which transmits and receives ultrasound using one of the plurality of ultrasound probes to generate ultrasound image data, the ultrasound diagnostic apparatus including: a signal-for-ultrasound generator which generates a signal for ultrasound and outputs the signal for ultrasound to the plurality of ultrasound probes in order; an ultrasound signal receiver which receives an ultrasound signal transmitted from the ultrasound probe in response to the signal for ultrasound; a detector which detects the ultrasound probe from which the ultrasound signal is received; and a hardware processor which switches and sets the detected ultrasound probe to be the ultrasound probe which is used.

According to another aspect, an ultrasound diagnostic apparatus to which a plurality of ultrasound probes are connected and which transmits and receives ultrasound using one of the plurality of ultrasound probes to generate ultrasound image data, the ultrasound diagnostic apparatus including: a signal-for-ultrasound generator which generates a signal for ultrasound; an ultrasound signal transmitter which transmits an ultrasound signal in response to the signal for ultrasound; a detector which detects the ultrasound probe which received the ultrasound signal; and a hardware processor which switches and the detected ultrasound probe to be the ultrasound probe which is used.

According to another aspect, an ultrasound probe switching method used in an ultrasound diagnostic apparatus to which a plurality of ultrasound probes are connected and which transmits and receives ultrasound using one of the plurality of ultrasound probes to generate ultrasound image data, the ultrasound probe switching method including: optically detecting the ultrasound probe which is used among the plurality of ultrasound probes; and switching and setting the detected ultrasound probe to be the ultrasound probe which is used.

According to another aspect, a non-transitory computer-readable storage medium storing a program causing a computer in an ultrasound diagnostic apparatus to which a plurality of ultrasound probes are connected and which transmits and receives ultrasound using one of the plurality of ultrasound probes to generate ultrasound image data, the program including: optically detecting the ultrasound probe which is used among the plurality of ultrasound probes; and switching and setting the detected ultrasound probe to be the ultrasound probe which is used.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present invention, and wherein:

FIG. 1 is a block diagram showing an ultrasound image management system according to an embodiment of the present invention;

FIG. 2 is a perspective view showing an outer appearance of an ultrasound diagnostic apparatus according to the present embodiment;

FIG. 3 is a block diagram showing a functional configuration of the ultrasound diagnostic apparatus according to the present embodiment;

FIG. 4 is a block diagram showing a functional configuration of a portable terminal;

FIG. 5 is a flowchart showing a first ultrasound probe switching process;

FIG. 6 is a diagram showing an ultrasound probe with an AR marker attached;

FIG. 7 is a diagram showing a scanning site selection by operation of the ultrasound probe;

FIG. 8 is a flowchart showing a communication access information setting process;

FIG. 9 is a flowchart showing an examination ID providing process;

FIG. 10 is a flowchart showing a capturing process;

FIG. 11 is a sequence diagram showing a moving image management process;

FIG. 12 is a diagram showing an outer appearance of an ultrasound diagnostic gel container;

FIG. 13 is a partial cross-sectional view of the ultrasound diagnostic gel container;

FIG. 14A is a schematic diagram showing the ultrasound diagnostic gel container according to the present embodiment in a first stage of use;

FIG. 14B is a schematic diagram showing the ultrasound diagnostic gel container according to the present embodiment in a second stage of use;

FIG. 14C is a schematic diagram showing the ultrasound diagnostic gel container according to the present embodiment in a third stage of use;

FIG. 15A is a schematic diagram showing a conventional ultrasound diagnostic gel container in a first stage of use;

FIG. 15B is a schematic diagram showing a conventional ultrasound diagnostic gel container in a second stage of use;

FIG. 15C is a schematic diagram showing a conventional ultrasound diagnostic gel container in a third stage of use;

FIG. 15D is a schematic diagram showing a conventional ultrasound diagnostic gel container in a fourth stage of use;

FIG. 16 is a block diagram showing a functional configuration of an ultrasound diagnostic apparatus according to a first modification;

FIG. 17 is a flowchart showing a second ultrasound probe switching process; and

FIG. 18 is a block diagram showing a functional configuration of the ultrasound diagnostic apparatus according to the second modification.

### DETAILED DESCRIPTION OF EMBODIMENTS

Hereinafter, one or more embodiments of the present invention will be described with reference to the drawings. However, the scope of the invention is not limited to the disclosed embodiments.

Embodiments and first and second modifications according to the present invention are described in detail in order with reference to the attached drawings. The present invention is not limited to the illustrated examples.

### 1. Embodiment

### 1-1. Apparatus Configuration

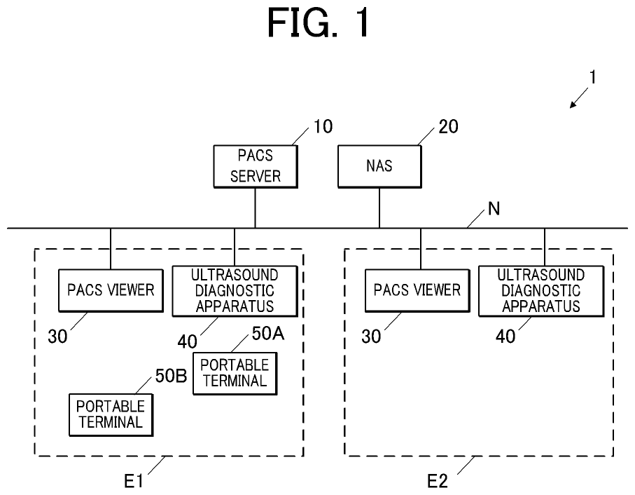

The embodiments of the present invention are described in detail with reference to FIG. 1 to FIG. 15D. First, the configuration of the entire apparatus according to the present embodiment is described with reference to FIG. 1. FIG. 1 is a block diagram showing a schematic configuration of the ultrasound image management system 1 according to the present embodiment.

The ultrasound image management system 1 according to the present embodiment is a system provided in a medical facility such as a hospital or a clinic. The ultrasound image management system captures the ultrasound image of the subject such as the live body of the patient and manages the ultrasound image data. As shown in FIG. 1, the ultrasound image management system 1 includes a Picture Archiving and Communication System (PACS) server 10, a Network Attached Storage (NAS) 20, a PACS viewer 30, an ultrasound diagnostic apparatus 40, and portable terminals 50A and 50B. The PACS server 10, the NAS 20, the PACS viewer 30, and the ultrasound diagnostic apparatus 40 are connected to each other through a communication network N.

The medical facility includes a first examination room E1 and a second examination room E2. The PACS viewer 30 and the ultrasound diagnostic apparatus 40 are provided in each of the first examination room E1 and the second examination room E2. The present embodiment described here is an example in which the patient and the person who performs the examination (doctor, examination technician, etc.) with the ultrasound diagnostic apparatus 40 enter the first examination room E1, and the patient is examined in the first examination room E1. The person who performs the examination holds the portable terminal 50A and the patient holds the portable terminal 50B.

The PACS server 10 is a server provided in the medical facility. The PACS server 10 mainly stores the radiation image data imaged with the radiation image imaging apparatus (not shown) and is able to store the ultrasound image data generated by the ultrasound diagnostic apparatus 40. The PACS server 10 provides the image data stored in the PACS server 10 to the PACS viewer 30 for viewing. There is a legal obligation to store the X-ray image data (still image data, moving image data) for five years, and the X-ray image data is stored in the PACS server 10. There is no obligation to hold the ultrasound image data. In order to reduce the consumption of the capacity of the PACS server 10, only the still image data of the ultrasound image is stored in the PACS server 10 because the still image data amount is relatively small.

The NAS 20 is a storage apparatus including a HDD (Hard Disk Drive), and a SSD (Solid State Drive). The NAS 20 is provided on the communication network N and the purpose of use is not limited. According to the present embodiment, the NAS 20 mainly stores the moving image data of the ultrasound image. Such moving image data has a relatively large amount of data. The NAS 20 is provided in the medical facility but a storage apparatus on a cloud such as a WAN (Wide Area Network) can be used. Preferably, the NAS 20 is a secure storage apparatus.

The PACS viewer 30 is an information processing apparatus as a client terminal of the PACS server 10. In response to the input of operation by the person who performs the examination, the PACS viewer 30 requests the image data stored in the PACS server 10 through the communication network N, obtains the image data from the PACS server 10 and displays the image data on the display for viewing.

The ultrasound diagnostic apparatus 40 is an apparatus which generates and stores the ultrasound image data of the subject. The ultrasound diagnostic apparatus 40 stores the ultrasound image data in its own apparatus. Through the communication network N, the ultrasound diagnostic apparatus 40 stores the still image data of the ultrasound image in the PACS server 10, and stores the moving image data of the ultrasound image in the NAS 20. The ultrasound diagnostic apparatus 40 includes an access point function of the wireless LAN (Local Area Network) such as Wi-Fi, and wireless LAN communication is possible with the portable terminal 50B.

The portable terminal 50A is a portable terminal including an image capturing function of the subject, and here, a smartphone is described as an example. The portable terminal 50A is not limited to a smartphone and can be a digital camera, a tablet PC (Personal Computer) or other terminal apparatus.

The portable terminal 50B is a portable terminal including a wireless LAN communication function, and here, a smartphone is described as an example. The portable terminal 50B is not limited to a smartphone and can be a tablet PC or other terminal apparatus.

The communication network N is a communication network such as a LAN provided in a medical facility. The communication network N is to be a wired LAN but can include other communication networks such as a wireless LAN.

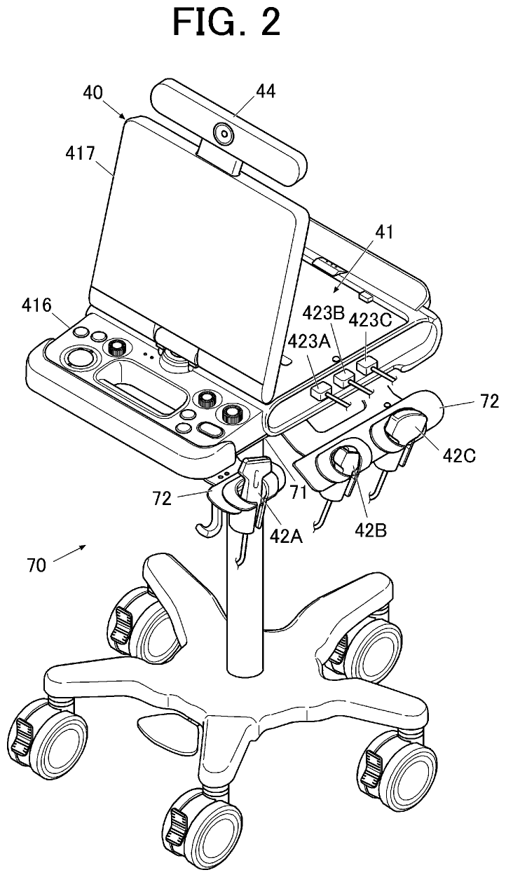

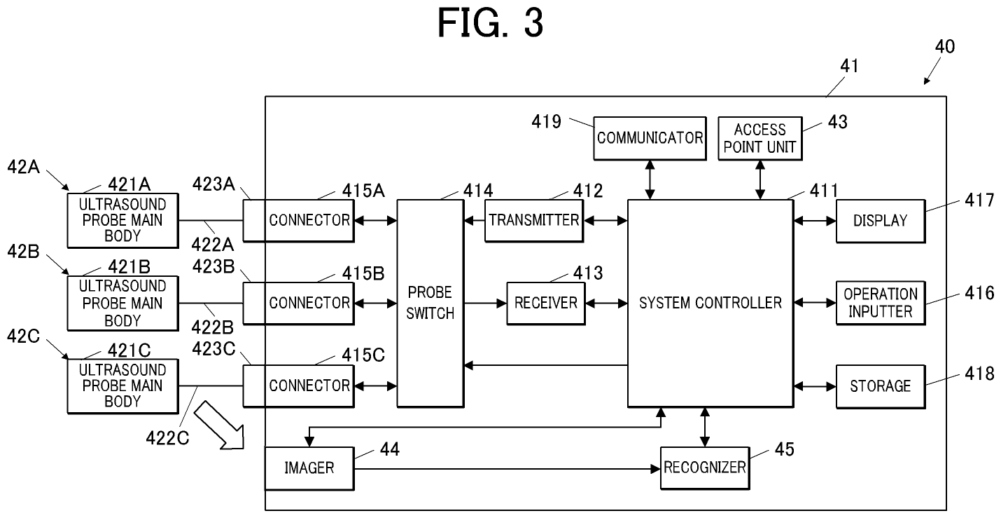

Next, the apparatus configuration of the ultrasound diagnostic apparatus 40 is described with reference to FIG. 2 and FIG. 3. FIG. 2 is a perspective view showing an outer appearance of the ultrasound diagnostic apparatus 40. FIG. 3 is a block diagram showing a functional configuration of the ultrasound diagnostic apparatus 40.

As shown in FIG. 2, the ultrasound diagnostic apparatus 40 includes an ultrasound diagnostic apparatus main body 41, and ultrasound probes 42A, 42B, and 42C. The ultrasound probes 42A, 42B, and 42C transmit ultrasound (transmitted ultrasound) to the subject such as the live body of the patient (not shown), and receive a reflected wave (reflected ultrasound: echo) of the ultrasound reflected on the subject. The ultrasound diagnostic apparatus main body 41 is connected to the ultrasound probes 42A, 42B, and 42C, and transmits driving signals as electric signals to the ultrasound probes 42A, 42B, and 42C to transmit the transmitted ultrasound to the subject. Based on the received signals which are electric signals generated in the ultrasound probes 42A, 42B, and 42C in response to the reflected ultrasound from the subject received with the ultrasound probes 42A, 42B, and 42C, the internal state of the subject is imaged as the ultrasound image.

The ultrasound probes 42A, 42B, and 42C are connected to the ultrasound diagnostic apparatus main body 41 at the same time, and when the ultrasound image is generated, one of the ultrasound probes is selected (switched) from the above and used.

The ultrasound probes 42A, 42B, and 42C include an oscillator (not shown) including a piezoelectric element. A plurality of such oscillators are arranged in a one-dimensional array in an orientation direction (scanning direction). The ultrasound probe 42A is an ultrasound probe which performs electronic scanning in a linear scanning method. The ultrasound probe 42B is an ultrasound probe which performs electronic scanning in a sector scanning method. The ultrasound probe 42C is an ultrasound probe which performs electronic scanning in a convex scanning method. However, the ultrasound probes 42A, 42B, and 42C are not limited to a combination of the linear scanning method, the sector scanning method, and the convex scanning method. For example, the ultrasound probes 42A, 42B, and 42C can include the plurality of ultrasound probes with the same type of scanning method and for different sites. The ultrasound probes 42A, 42B, and 42C can include other types of ultrasound probes such as the ultrasound probe in which the oscillators are arranged in a two-dimensional matrix.

Further, the number of ultrasound probes connected to the ultrasound diagnostic apparatus main body 41 at the same time is not limited to three and can be two or four or more.

The ultrasound diagnostic apparatus 40 is attached to a cart 70. The cart 70 is a car which can be moved by wheels. The cart 70 includes an ultrasound diagnostic apparatus attaching surface 71 and three holders 72. The ultrasound diagnostic apparatus attaching surface 71 is a flat surface where the ultrasound diagnostic apparatus main body 41 is attached. The three holders 72 are holders which hold the ultrasound probes 42A, 42B, and 42C.

As shown in FIG. 2 and FIG. 3, the ultrasound diagnostic apparatus main body 41 includes a system controller 411 as the setter, a transmitter 412, a receiver 413, a probe switch 414, connectors 415A, 415B, and 415C, an operation inputter 416, a display 417, a storage 418, a communicator 419, an access point unit 43, an imager 44, and a recognizer 45. The imager 44 and the recognizer 45 function as the detector.

The system controller 411 functions as the hardware processor and includes a CPU (Central Processing Unit), a ROM (Read Only Memory), and a RAM (Random Access Memory). The CPU reads various processing programs such as a system k stored in the ROM, deploys the program in the RAM and controls various units of the ultrasound diagnostic apparatus 40 according to the deployed program. The ROM includes a nonvolatile memory such as a semiconductor. The ROM stores a system program corresponding to the ultrasound diagnostic apparatus 40 and various processing programs which can be executed on the system program such as an ultrasound image display program to execute a later-described ultrasound image display process and various data such as a gamma table. Such programs are stored in a state as program codes which can be read by the computer, and the CPU sequentially executes the operation according to the program code. The RAM forms a work area which temporarily stores various programs executed by the CPU and data regarding the various programs.

The ROM of the system controller 411 stores a first ultrasound probe switching program which executes a later-described first ultrasound probe switching process, a communication access information setting program which executes a later-described communication access information setting process, an examination ID providing program which executes a later-described examination ID providing process, and a moving image storage program which executes a later-described moving image storage process.

According to the control by the system controller 411, the transmitter 412 is a circuit which supplies a driving signal which is an electric signal to the ultrasound probes 42A, 42B, and 42C through the probe switch 414 and connectors 415A, 415B, and 415C, and generates the transmitted ultrasound in the ultrasound probes 42A, 42B, and 42C. The transmitter 412 includes a clock generating circuit, a delaying circuit, a pulse generating circuit or a calculating circuit including the above functions. The clock generating circuit is a circuit which generates a clock signal which determines the transmitting timing of the driving signal and the transmitting frequency. The delay circuit is a circuit which sets transmitting timing of the driving signal as the delay time for each individual path corresponding to each piezoelectric circuit, delays the transmitting of the driving signal in the amount of the set delay time, and focuses the transmitted beam including the transmitted ultrasound. The pulse generating circuit is a circuit which generates a pulse signal as the driving signal in a predetermined cycle. The transmitter 412 as described above drives some of the continuous ones among the plurality of oscillators arranged in the ultrasound probes 42A, 42B or 42C and generates the transmitted ultrasound.

According to the control by the system controller 411, the receiver 413 is a circuit which receives received signals which are electric signals from the ultrasound probes 42A, 42B, and 42C through the probe switch 414 and the connectors 415A, 415B, and 415C. The receiver 413 includes an amplifier, an A/D converting circuit, and a phase adjusting adder circuit. The amplifier is a circuit which amplifies the received signal at a pre-set amplifying rate for each individual path corresponding to each piezoelectric element. The A/D converting circuit is a circuit for A/D conversion of the amplified received signal. The phase adjusting adder circuit is a circuit which provides delay time to the received signal after A/D conversion for each individual path corresponding to each piezoelectric element in order to adjust the time phase, and adds (phase adjusting and adding) the above to generate sound ray data.

The probe switch 414 is a switching circuit connected to the transmitter 412, the receiver 413, and the connectors 415A, 415B, and 415C. According to the control by the system controller 411, the probe switch 414 selects one of the connectors 415A, 415B, and 415C and switches the electric connection to the ultrasound probe as the transmitting destination of the driving signals transmitted by the transmitter 412. The probe switch 414 also selects one of the connectors 415A, 415B, and 415C and switches the electric connection from the ultrasound probe as the source of the received signal received by the receiver 413.

The connector 415A is a connector physically and electrically connected to a connector 423A of the ultrasound probe 42A. The connector 415B is a connector physically and electrically connected to a connector 423B of the ultrasound probe 42B. The connector 415C is a connector physically and electrically connected to a connector 423C of the ultrasound probe 42C.

The ultrasound probe 42A includes an ultrasound probe main body 421A, a cable 422A, and a connector 423A. The ultrasound probe main body 421A is the portion where the ultrasound is transmitted and received between the subject. The cable 422A is a cable electrically connecting the ultrasound probe main body 421A and the connector 423A. The connector 423A is a connector which physically and electrically connects the ultrasound probe 42A to the connector 415A, 415B or 415C, and includes a storage to store identification information of the ultrasound probe 42A. Therefore, the system controller 411 reads the identification information of the ultrasound probe 42A from the storage of the connector 423A, and it is possible to recognize which connector (connector 415A, 415B, or 415C) the ultrasound probe 42A is connected to.

Similar to the ultrasound probe 42A, the ultrasound probe 42B includes the ultrasound probe main body 421B, the cable 422B, and the connector 423B. Similarly, the ultrasound probe 42C includes the ultrasound probe main body 421C, the cable 422C, and the connector 423C.

The system controller 411 performs an envelope detection process or a log compression on sound ray data from the receiver 413, performs the adjustment of dynamic range and gain, and converts the brightness. With this, B (brightness) mode image data is generated. The B mode image data is tomographic image data as the ultrasound image data. That is, B mode image data shows the strength of the received signal with brightness. The system controller 411 may be able to generate ultrasound image data in another image mode such as color Doppler mode in addition to B mode image data in the B mode.

The system controller 411 performs various information processes on the generated ultrasound image data and displays on the display 417 the ultrasound image data on which the information process is performed in a unit of frames, and when the storage instruction is input from the person who performs the examination through the operation inputter 416, the system controller 411 stores the generated ultrasound image data (still image data, moving image data) in the storage 418.

The operation inputter 416 includes various switches, various keys (hard key), a track ball, a mouse, and the like to perform input of a command to instruct the start of diagnosis or input of data such as personal information of the subject. The operation inputter 416 outputs to the system controller 411 an operation signal in response to the input of the operation from the person who performs the examination The operation inputter 416 may include a touch panel formed on a display screen of the display 417. In this case, the operation inputter 416 receives the touch operation from the person who performs the examination and outputs the touch operation information to the system controller 411.

As the display 417, display apparatuses such as an LCD (Liquid Crystal Display), an organic EL (Electronic Luminescence) display, an inorganic EL display, and a plasma display can be applied. According to the display information from the system controller 411, the display 417 displays the display information such as the ultrasound image on the display screen.

The storage 418 includes a large capacity storage medium such as a HDD, or a SSD and stores the data such as the ultrasound image data. The storage 418 includes a public folder for each patient to store data made public to the portable terminal 50B as the connection destination of the wireless LAN communication through the access point unit 43.

The communicator 419 includes a network card connected to the communication network N, and transmits and receives information between the device on the communication network. The system controller 411 performs communication with devices such as the PACS server 10 and NAS 20 on the communication network N through the communicator 419.

The access point unit 43 includes an antenna, a modulator/demodulator, and a signal processor, and includes a function to transmit and receive information in a wireless LAN format to relay the information. According to the control by the system controller 411, a SSID (Service Set Identifier: identification name of access point) and a password which can be changed can be set in the access point unit 43. Through the access point unit 43, the system controller 411 performs wireless communication by the wireless LAN communication format with the terminal apparatus (for example, portable terminal 50B) in which the SSID and the password set in the access point unit 43 is set.

For example, the imager 44 is a digital camera which is provided above the display 417 and with which the target can be imaged. The imager 44 includes an optical system, an imaging element, and the like. According to the control by the system controller 411, the imager 44 images the subject (patient) as a target, generates image data, and outputs the generated image data to the recognizer 45.

According to the control by the system controller 411, the recognizer 45 analyzes the image data input from the imager 44 and outputs an analysis result to the system controller 411. This image analysis is the image analysis for the identification information of the ultrasound probe used by the person who performed the examination (can also be an identifier such as an AR (Augmented Reality) code attached to the ultrasound probe or external shape of ultrasound probe) from the imaged image data.

Regarding each unit included in the ultrasound diagnostic apparatus 40, some or all of the functions in each functional block can be realized as a hardware circuit such as an integrated circuit. For example, the integrated circuit is a LSI (Large Scale Integration), and due to the difference of the degree of integration, the LSI may be called an IC (integrated Circuit), a system LSI, a super LSI or an ultra LSI. The method of integrated circuitry is not limited to an LSI, and can be a dedicated circuit or a general processor. A reconfigurable processor in which the connection and setting in the circuit cell in the FPGA (Field Programmable Gate Array) or the LSI can be reconfigured can be used. Some or all of the functions of the functional block can be executed by software. In this case, the software is stored in one or more among a storage medium such as a ROM, an optical disk or a hard disk, and the software is executed by a computing processor.

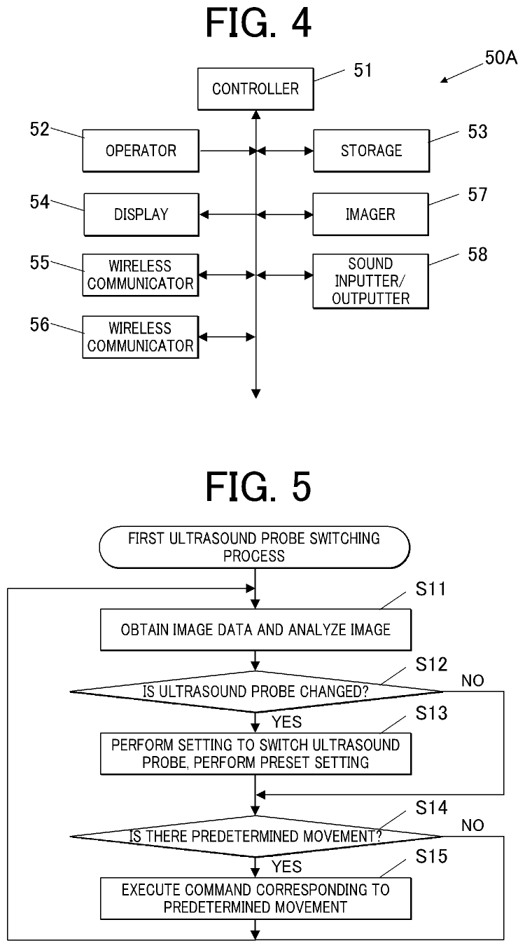

Next, the functional configuration in the portable terminal 50A is described with reference to FIG. 4. FIG. 4 is a block diagram showing a functional configuration of the portable terminal 50A.

As shown in FIG. 4, the portable terminal 50A includes a controller 51, an operator 52, a storage 53, a display 54, a wireless communicator 55 and 56, an imager 57, a sound inputter/outputter 58 and the like. Each unit of the portable terminal 50A are connected to each other through a bus.

The controller 51 includes a CPU, a RAM, and a ROM, and controls each unit of the portable terminal 50A. The CPU of the controller 51 reads the program specified among the various programs from the ROM and deploys the program in the RAM. In coordination with the deployed program, the CPU of the controller 51 executes various processes. The ROM stores the capturing program which executes a later-described capturing process.

For example, the operator 52 includes a touch panel formed as one with the display screen of the display 54 and various hard keys. The operator 52 receives the input of operation from the person who performs the examination, and outputs the operation signal according to the input operation to the controller 51.

The storage 53 is a nonvolatile semiconductor memory in which information is readable and writable so that various information is stored. Examples include a flash memory.

The display includes a display panel such as an LCD, an organic EL display, or the like, and displays on the display panel an image based on the display control signal output from the controller 51.

The wireless communicator 55 is a communicator in a mobile communication format, and includes an antenna in a mobile communication format, a modulator/demodulator, and a signal processor. The controller 51 uses the wireless communicator 55 to perform communication with external devices connected to a base station through wireless communication with the base station.

The wireless communicator 56 is a communicator in a wireless LAN communication format and includes an antenna in a wireless LAN communication format, a modulator/demodulator, and a signal processor. The controller 51 uses the wireless communicator 56 to perform communication with external devices connected to an access point through wireless communication with the access point.

The imager 57 includes an optical system, an imaging element and the like. According to control by the controller 51, the imager 57 images a subject (patient) as a target, generates image data, and outputs the generated image data to the controller 51 or the storage 53.

The sound inputter/outputter 58 functions as a sound inputter including a microphone, etc., and according to control by the controller 51, the sound inputter/outputter 58 receives input of the voice of the person who performs the examination and obtains sound data. The sound inputter/outputter 58 functions as a sound output unit including a D/A converter, amplifier, and speaker. The sound inputter/outputter 58 converts the sound data output from the controller 51 to an analog sound signal and outputs the sound.

The portable terminal 50A may include other components such as a position detector of a satellite positioning system. The configuration of the portable terminal 50B is similar to the configuration of the portable terminal 50A. The operator of the portable terminal 50B is the patient.

### 1-2. Operation of Ultrasound Image Management System 1

Next, various operations of the ultrasound image management system 1 are described with reference to FIG. 5 to FIG. 15B.

### 1-2-1. First Ultrasound Probe Switching Process

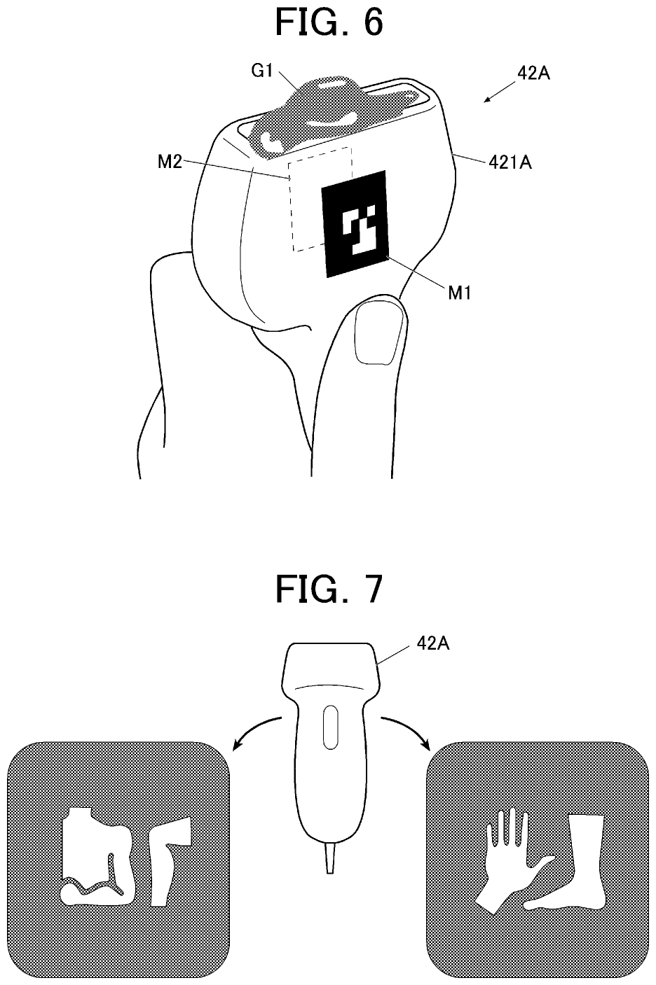

The first ultrasound probe switching process executed by the ultrasound diagnostic apparatus 40 is described with reference to FIG. 5 to FIG. 7. FIG. 5 is a flowchart showing the first ultrasound probe switching process. FIG. 6 is a diagram showing an ultrasound probe 42A with the AR marker M1 attached. FIG. 7 is a diagram showing a scan site selection by operation of the ultrasound probe 42A.

For example, in the first examination room E1, the ultrasound diagnostic apparatus 40 is used and the examinations for a plurality of patients are performed in order. In the first examination room E1, the person who performs the examination enters the room, the patient enters the room, and for each examination, the patient leaves the room after the examination ends, and the next patient enters the room. The ultrasound probes 42A, 42B, and 42C are connected to the ultrasound diagnostic apparatus main body 41 of the ultrasound diagnostic apparatus 40 in the first examination room E1. An AR marker as the unique identifier including the identification information (type) of the ultrasound probe is attached to each of the ultrasound probes 42A, 42B, and 42C. For example, as shown in FIG. 6, the AR marker M1 is attached to the surface of the ultrasound probe main body 421A of the ultrasound probe 42A. The ultrasound diagnostic gel G1 is attached to the ultrasound transmitting/receiving portion (acoustic lens) at the tip of the ultrasound probe 42A.

The AR marker M1 includes identification information of the ultrasound probe 42A, and identification information showing the depth of the ultrasound image is deep as preset contents of the examination On the back side of the ultrasound probe main body 421A, the AR marker M2 is attached. The AR marker M2 includes the identification information of the ultrasound probe 42A, and identification information showing the depth is shallow as preset contents of the examination The preset contents of the examination can be other contents such as the frequency of the ultrasound. Such AR marker is not related to the display of the AR.

In the examination, the person who performs the examination operates the ultrasound diagnostic apparatus 40, and performs the scanning for the ultrasound image of the subject. During the examination, when the used ultrasound probe is changed, the person who performs the examination holds the changed ultrasound probe and shows the probe to the imager 44 so that the AR marker is imaged.

In the ultrasound diagnostic apparatus 40, for example, the instruction to execute the first ultrasound probe switching process input through the operation inputter 416 from the person who performs the examination functions as a trigger and the system controller 411 performs the first ultrasound probe switching process according to the first ultrasound probe switching program stored in the ROM.

As shown in FIG. 5, first, the system controller 411 obtains the image data (moving image data) of the plurality of frames imaged through the imager 44, allows the recognizer 45 to analyze the image of the obtained image data, and obtains the analysis result (step S11). Here, the recognizer 45 analyzes the image of the AR marker in the obtained image data. The recognizer 45 generates the analysis result showing whether the AR marker is imaged, and when the AR marker is imaged, the identification information of the ultrasound probe and the preset contents of the examination from the AR marker.

The AR marker attached to the ultrasound probe may be different between the AR marker including the identification information of the ultrasound probe and the AR marker including the preset contents of the examination. As the identifier attached to the ultrasound probe, the AR marker is not readable to the person who performs the examination but alternatively, the AR marker may be readable. As the identifier attached to the ultrasound probe, other identifiers can be used such as symbols including a one-dimensional barcode or two-dimensional barcode or character information such as a model number. The recognizer 45 in step S11 can analyze the image of the external shape itself of the ultrasound probe to obtain the identification information of the ultrasound probe.

According to the image analysis in step S11, the recognizer 45 analyzes whether there is a predetermined movement (gesture) of the ultrasound probe set in advance, and this is included in the analysis result. The predetermined movement of the ultrasound probe includes, for example, as shown in FIG. 7, the movement to lay the ultrasound probe 42A toward the left or the movement to lay the ultrasound probe 42A toward the right with relation to the imager 44. Here, the movement to lay the ultrasound probe 42A to the left may be corresponded to the command to perform setting that the scanning site is the arm or the leg, and the movement to lay the ultrasound probe 42A to the right may be corresponded to the command to perform the setting that the scanning site is the hand or the tip of the foot.

However, the predetermined movement of the ultrasound probe and the command in the ultrasound diagnostic apparatus 40 corresponded with the above is not limited to the above example. For example, when the AR marker M1 is imaged, and the preset contents show the depth is shallow, the movement to lay the ultrasound probe 42A to the left corresponds to the command to set the depth to 2 cm and the movement to lay the ultrasound probe 42A to the right corresponds to the command to set the depth to 5 cm.

Then, according to the analysis result in step S11, the system controller 411 determines whether to change the ultrasound probe which is used (step S12). When the used ultrasound probe is changed (step S12; YES), according to the analysis result in step S11, the system controller 411 uses the probe switch 414 to perform the switch setting to electrically connect the path corresponding to the changed ultrasound probe as the path for the driving signal and the received signal, and to perform the setting of the preset contents of the examination (step S13). When the identification information of the ultrasound probe which is used is specified, the connector 415A, 415B, or 415C to which the ultrasound probe is connected is specified by the identification information in the storage for the connector 423A, 423B, or 423C, and the switch setting is possible.

Then, according to the analysis result in step S11, the system controller 411 determines whether there is a predetermined motion (step S14). When there is no change in the ultrasound probe which is used (step S12; NO), the process progresses to step S14. When there is a predetermined movement (step S14; YES), the system controller 411 executes the command corresponding to the predetermined movement (step S15), and the process progresses to step S11. When there is no predetermined movement (step S14; NO), the process progresses to step S11.

As described above, according to the first ultrasound probe switching process, the ultrasound diagnostic apparatus 40 is an ultrasound diagnostic apparatus in which the ultrasound probe 42A, 42B, and 42C are connected, and one of the ultrasound probes 42A, 42B, and 42C is used to transmit and receive the ultrasound. With this, the ultrasound image data is generated. The ultrasound diagnostic apparatus 40 includes the imager 44 and the recognizer 45 which optically detects the ultrasound probe to be used among the ultrasound probes 42A, 42B, and 42C and a system controller 411 which switches and sets the detected ultrasound probe to be the ultrasound probe which is used. Therefore, an existing ultrasound probe is used to place the ultrasound probe desired to be used over the imager 44 of the ultrasound diagnostic apparatus 40 and the ultrasound probe to be used can be switched easily.

The imager 44 and the recognizer 45 images the identifier such as the AR marker M1 and M2 including the identification information of the ultrasound probe attached to the ultrasound probe, and detects the ultrasound probe of the identification information included in the identifier. Therefore, the used ultrasound probe can be easily and reliably detected.

The imager 44 and the recognizer 45 image the ultrasound probe and detect the ultrasound probe from the external shape of the ultrasound probe. Therefore, the ultrasound probe to be used can be easily detected without attaching anything to the existing ultrasound probe.

The imager 44 and the recognizer 45 image the identifier such as the AR marker M1 and M2 attached to the ultrasound probe, the identifier including the preset contents of the examination using the ultrasound probe, and detect the preset contents of the examination included in the identifier. The system controller 411 sets the detected preset contents of the examination. Therefore, the preset contents of the ultrasound probe to be used can be set easily and reliably.

The imager 44 and the recognizer 45 image the ultrasound probe and detect the predetermined movement of the ultrasound probe. The system controller 411 executes the command corresponding to the detected predetermined movement. Therefore, in response to the predetermined movement of the ultrasound probe by the person who performs the examination, the corresponding command can be easily executed.

### 1-2-2. Communication Access Information Setting Process

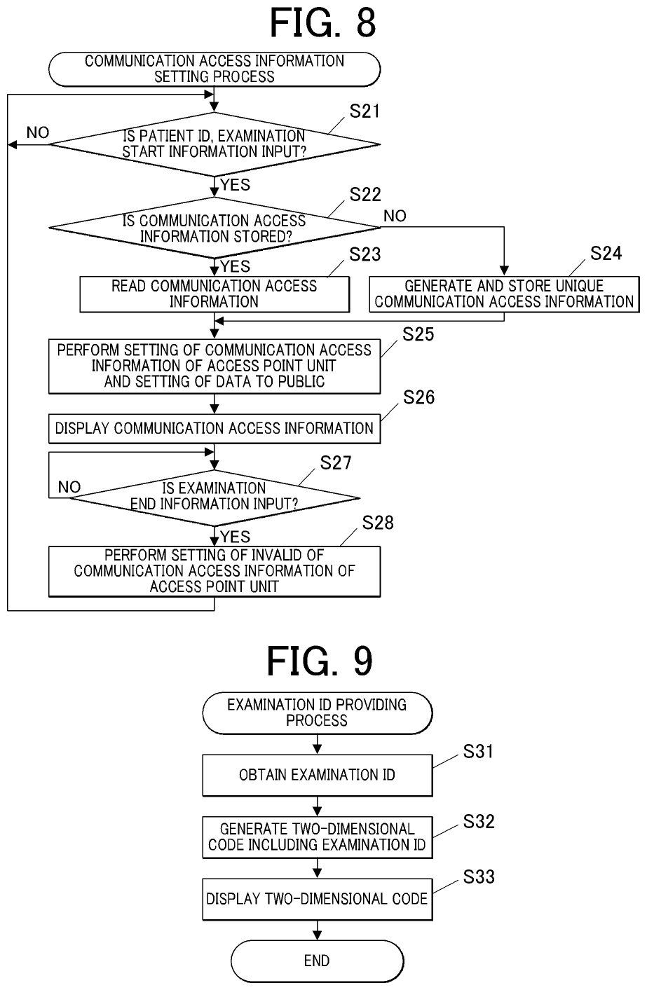

With reference to FIG. 8, the communication access information setting process executed in the ultrasound diagnostic apparatus 40 is described. FIG. 8 is a flowchart showing a communication access information setting process.

Conventionally, there are cases in which the person who performs the examination provides the data to the patient after the examination. The data provided to the patient include, ultrasound image data (for example, fetus), report (letters and graphs, may include the ultrasound image) of the examination result (for example, examination of arteriosclerosis), and interview data. For example, there is a well-known ultrasound diagnostic apparatus on which the barcode is printed on the sheet including the URL (Uniform Resource Locator) information to access to the data storage server storing data (see JP 2013-312). There is a well-known ultrasound diagnostic apparatus in which a two-dimensional code including URL information to access to an image management apparatus in which data is stored is printed on a sheet (JP 2017-182244).

However, according to the ultrasound diagnostic apparatus described in JP 2013-312 and JP 2017-182244, if the Internet is used, safety needs to be secured in the communication path such as by VPN (Virtual Private Network). Therefore, it is necessary to provide the data by a local network. On the other hand, if the terminal apparatus of the patient connects to the local network of the hospital, there is the risk of leak of information and increase in the network load, and therefore, it is not preferable. It is inconvenient for the patient to connect to the network in the hospital in which Internet connection is not possible.

The problems to be solved by the present embodiment corresponding to the above items is to provide a local communication environment in which only the patient who is examined is able to access to the ultrasound diagnostic apparatus.

Therefore, according to the present embodiment, the ultrasound diagnostic apparatus 40 includes an access point unit 43 for P2P (point to point) connection with the portable terminal 50B of the patient, and only the patient is able to access to the data in the examination room after the examination The range in which the communication by the access point unit 43 is possible at least includes inside the examination room in which the ultrasound diagnostic apparatus 40 is provided. During the examination and after the examination, the system controller 411 creates data to be provided to the patient based on the ultrasound image data obtained in the examination, the image analysis result of the ultrasound image data, and the input information from the person who performs the examination received on the operation inputter 416. The system controller 411 stores the data in a public folder corresponding to the patient ID of the patient in the storage 418.

Here, the patient and the person who performs the examination enters the first examination room E1, and the examination including the scanning for the ultrasound image of the subject using the ultrasound diagnostic apparatus 40 is performed. After the examination, the next patient enters after the patient whose examination is finished leaves the room and the next examination is started.

According to the ultrasound diagnostic apparatus 40, for example, when the person who performs the examination inputs the execution instruction of the communication access information setting process through the operation inputter 416, this triggers the system controller 411 to perform the communication access information setting process according to the communication access information setting program stored in the ROM. The SSID and the password for the wireless LAN communication by the access point unit 43 is set to be invalid so that wireless LAN communication cannot be performed.

As shown in FIG. 8, the system controller 411 determines whether the patient ID of the patient who is examined by the person who performs the examination and the examination start information are input through the operation inputter 416 (step S21). When the patient ID and the examination start information are not input (step S21; NO), the process progresses to step S21.

When the patient ID and the examination start information are input (step S21; YES), the system controller 411 determines whether the communication access information (SSID and password) corresponding to the patient ID input in step S21 is stored in the storage 418 (step S22). When the communication access information is stored (step S22; YES), the system controller 411 reads the communication access information corresponding to the patient ID input in step S21 from the storage 418 (step S23).

When the communication access information is not stored (step S22; NO), the system controller 411 generates the unique communication access information corresponding to the patient ID input in step S21, corresponds this to the patient ID, and stores the information in the storage 418 (step S24).

Then, the system controller 411 sets the communication access information of the access point unit 43 as the communication access information obtained in step S23 or S24, and sets the public folder for the patient ID input in step S21 to public (step S25). Then, the system controller 411 displays the communication access information set in step S25 on the display 417 (step S26). The communication access information displayed in step S26 may only be displayed when the person who performs the examination inputs an instruction to display through the operation inputter 416 in a position which does not interfere with the examination, for example, an edge of a display screen. According to step S26, only the person inside the first examination room E1 (patient, person who performs the examination) can confirm the communication access information by sight.

The communication access information displayed in step S26 can be letter information such as the SSID and the password or coded information such as a two-dimensional code “QR Wi-Fi” including the SSID and the password. For example, when the two-dimensional code of the communication access information is displayed, the patient places the imager 57 of the portable terminal 50B over the display 417. In response to the input operation by the patient through the operator 52, the controller 51 of the portable terminal 50B images the two-dimensional code with the imager 57. The controller 51 decodes the image data of the imaged two-dimensional code and obtains the communication access information. The controller 51 uses the obtained communication access information and accesses by the wireless LAN method to the access point unit 43 of the ultrasound diagnostic apparatus 40 through the wireless communicator 56. Then, the controller 51 suitably downloads the data which is stored in the public folder in the storage 53 and which is made public, and displays the data on the display 54. The data is stored in the storage 53.

Then, the system controller 411 determines whether the examination end information is input from the person who performs the examination through the operation inputter 416 (step S27). When the examination end information is not input (step S27; NO), the process progresses to step S27. For example, after the present patient leaves the first examination room E1, the examination end information is input.

When the examination end information is input (step S27; YES), the system controller 411 sets the communication access information set in the access point unit 43 to invalid (step S28) and the process progresses to step S21. Therefore, the access to the access point unit 43 becomes invalid for the patient who leaves from the first examination room after the examination ends. Therefore, it is possible to prevent access from third parties with bad intentions or to prevent access from other patients resulting in accidentally viewing the patient's data.

In the next examination and after, the patient who accessed to the ultrasound diagnostic apparatus 40 in the first examination is able to automatically access to the access point unit 43 of the ultrasound diagnostic apparatus 40 in the room without entering the communication access information again (letter input, two-dimensional code imaging, etc.) if the patient has the same portable terminal 50B.

According to the above configuration, if a plurality of examinations are performed on the same patient, it is assumed that the same examination room is used, but the situation is not limited to the above. If the patient ID and communication access information and the public folder for each patient ID are shared by the plurality of ultrasound diagnostic apparatuses 40 in a plurality of examination rooms, even if different examination rooms are used for a plurality of examinations on the same patient, the patient is able to access to his past data.

As described above, according to the communication access information setting process, the communication environment of the wireless LAN corresponding to the communication access information is provided and the communication access information is displayed only during the examination. Therefore, the local communication environment is provided so that only the patient who is examined is able to access to the ultrasound diagnostic apparatus. The public folder corresponding to the communication access information is made public and therefore, the data corresponding to the patient can be reliably provided only when the patient who is examined accesses to the ultrasound diagnostic apparatus. When the examination ends, the communication environment of the wireless LAN corresponding to the communication access information is stopped. Therefore, it is possible to reliably prevent the patient other than the patient who is examined or a third party having access to the data of the examined patient on purpose or by accident. Further, the communication access information is stored corresponded with the patient ID of the examined patient. Therefore, the burden of inputting communication access information is decreased for the examined patient from the second time and after.

### 1-2-3. Examination ID Providing Process

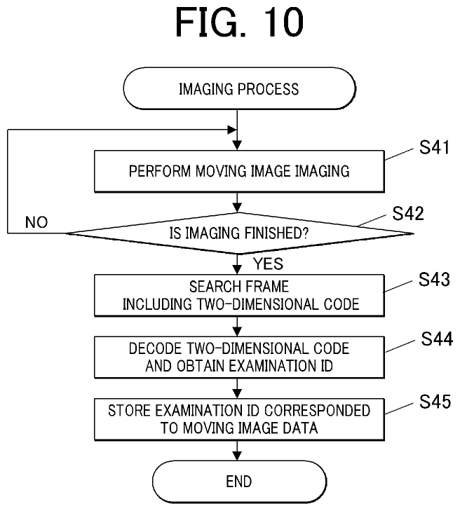

With reference to FIG. 9 and FIG. 10, the examination ID providing process executed by the ultrasound diagnostic apparatus 40 is described. FIG. 9 is a flowchart showing the examination ID providing process. FIG. 10 is a flowchart showing the capturing process.

In the examination including scanning for the ultrasound image, depending on the person who performs the examination, a portable terminal such as a smartphone different from the ultrasound diagnostic apparatus is used to capture the moving image of the patient as the subject, stores the captured moving image data in his PC and uses the data as academic material for presentations in an academic society.

The imager is provided in the terminal apparatus such as a tablet PC which functions as the ultrasound diagnostic apparatus, and the ultrasound image data and the moving image data of the captured subject may be stored corresponded to each other. However, it may be easier to capture and easier to obtain the intended moving image data by performing the capturing using the portable terminal different from the ultrasound diagnostic apparatus.

However, the ultrasound diagnostic apparatus and the portable terminal are different apparatuses, and it is a heavy burden for the person who performs the examination to manually input the examination ID of the examination being executed each time and corresponding this with the captured moving image data.

The problem to be solved by the present embodiment corresponding to the above items is to easily correspond the examination ID to the captured moving image data.

According to the present embodiment, the ultrasound diagnostic apparatus 40 displays the examination ID and provides the examination ID to the person who performs the examination.

Here, the following situation is already in progress. Both the patient and the person who performs the examination entered the first examination room E1 and the examination including the scanning for the ultrasound image of the subject using the ultrasound diagnostic apparatus 40 is performed. The system controller 411 of the ultrasound diagnostic apparatus 40 in the first examination room E1 receives the input of the examination ID of the examination which is executed at present from the person who performs the examination through the operation inputter 416 and performs the ultrasound image display process which displays on the display 417 the ultrasound image data obtained by the scanning for the ultrasound image. For example, the input examination ID is stored in the storage 418.

First, with reference to FIG. 9, the examination ID providing process executed in the ultrasound diagnostic apparatus 40 of the first examination room E1 is described. In the ultrasound diagnostic apparatus 40, for example, the person who performs the examination inputting the instruction to execute the examination ID providing process through the operation inputter 416 functions as a trigger and the system controller 411 performs the examination ID providing process according to the examination ID providing program stored in the ROM.

As shown in FIG. 9, first, the system controller 411 reads the examination ID of the examination being executed at present and obtains the examination ID from the storage 418 (step S31). Then, the system controller 411 generates the image data of the two-dimensional code including step S31 (step S32).

Then, the system controller 411 displays on the display 417 the image data of the two-dimensional code generated in step S32 (step S33) and ends the examination ID providing process. In step S33, the edge of the display screen of the display 417 may be displayed so as not to interfere with the examination

Next, with reference to FIG. 10, the capturing process executed by the portable terminal 50A held by the person performing the examination is described. According to the portable terminal 50A, for example, the input of the instruction to execute the capturing process from the person who performs the examination through the operator 52 functions as the trigger and the controller 51 performs the capturing process according to the capturing program stored in the ROM.

As shown in FIG. 10, in response to input regarding the capturing from the person who performs the examination through the operator 52, the controller 51 controls the imager 57 to image the moving image of the patient as the subject and stores the imaged moving image data in the storage 53 (step S41). In step S41, the person who performs the examination performs the moving image capturing pointing the imager 57 to the display 417 so that the two-dimensional barcode being displayed in the examination ID providing process is imaged in at least one of the frames of the captured moving image data.

Then, the controller 51 determines whether the instruction to end the capturing is input from the person who performs the examination through the operator 52 and the moving image capturing is to end (step S42). When the moving image capturing does not end (step S42; NO), the process progresses to step S41. When the moving image capturing ends (step S42; YES), the controller 51 analyzes the image of each frame in the moving image data stored in the storage 53 and searches for the frame including the image of the two-dimensional code (including the examination ID) (step S43).

Then, the controller 51 decodes the image of the two-dimensional code of the frame searched in step S43 and obtains the examination ID (step S44). Then, the controller 51 stores the moving image data captured in step S41 in the storage 53 corresponded with the examination ID obtained in step S44 (step S45) and ends the capturing process.

As described above, according to the examination ID providing process, the person who performs the examination is able to easily correspond the examination ID to the captured moving image data by using the portable terminal 50A.

According to the above configuration, the portable terminal 50A finds the two-dimensional code from the frame of the captured moving image data and decodes the code but the present invention is not limited to the above. The controller 51 of the portable terminal 50A may transmit the moving image data including the captured two-dimensional code to the ultrasound diagnostic apparatus 40 through the wireless communicator 56. The system controller 411 can find the two-dimensional code from the frame of the moving image data received through the access point unit 43 and decode the code. The system controller 411 can store in the storage 418 the ultrasound image data corresponding to the examination ID and the received image data so that the ultrasound image data is corresponded with the received image data by using the decoded examination ID. Alternatively, for example, the controller 51 of the portable terminal 50A can transmit the moving image data including the captured two-dimensional code through the wireless communicator 55 or 56 and the communication network N to the storage apparatus such as the PACS server 10 and NAS 20. The controller of the storage apparatus can find the two-dimensional code from the frame of the received moving image data and decode the code, and store in its storage the ultrasound image data corresponding to the examination ID and the received image data so that the ultrasound image data is corresponded with the received image data by using the decoded examination ID.

According to the above configuration, the examination ID is included in the moving image data, but the configuration is not limited to the above. The two-dimensional code including the examination ID can be displayed on the display 417, and if there is still image data capturing the subject and the two-dimensional code in one frame at the same time with the portable terminal 50A, similar to the above examination ID providing process and capturing process, the still image data can be stored in the storage 53 corresponded with the examination ID.

### 1-2-4. Moving Image Management Process

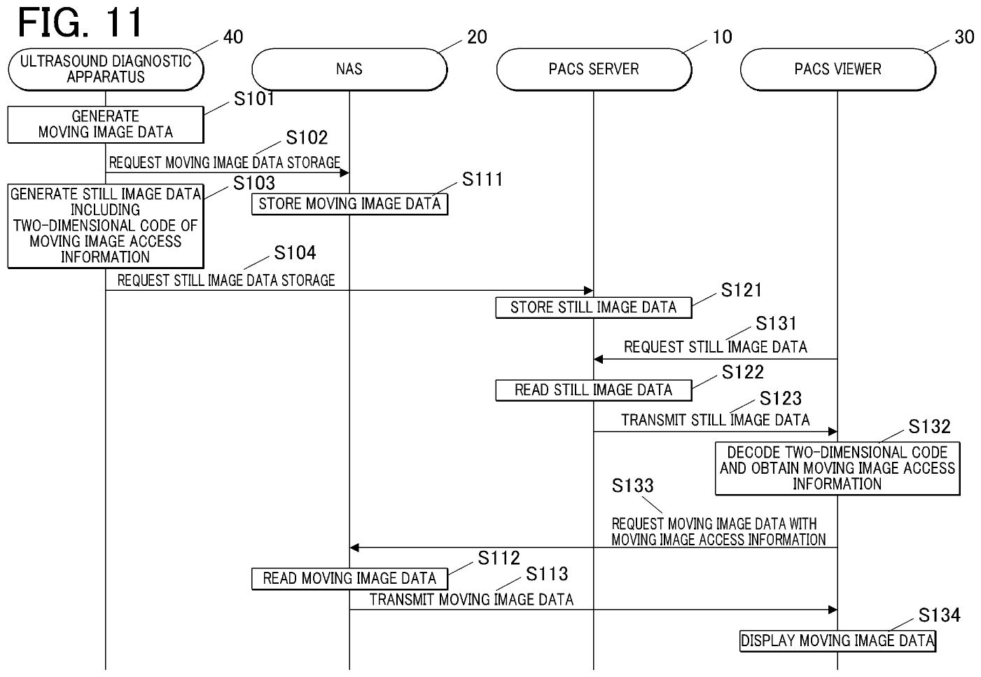

The moving image management process performed in the ultrasound image management system 1 is described with reference to FIG. 11. FIG. 11 is a sequence diagram showing the moving image management process.

As described in (1-1), the radiation image data is stored in the PACS server because there is an obligation to store the radiation image data. The ultrasound image data is also stored in the PACS server for the purpose of convenience.

As the apparatus which accesses to the stored image data, there is a well-known ultrasound diagnostic apparatus in which a barcode is printed on a sheet and the barcode includes the URL information to access to the data storage server in which the data is stored (see JP 2013-312). There is also a well-known ultrasound diagnostic apparatus in which a two-dimensional code is printed on a sheet and the two-dimensional code includes URL information to access to the image management apparatus in which data is stored (see JP 2017-182244).

However, the moving image data of the ultrasound image which consumes a large amount of the capacity is not stored in the PACS server from the viewpoint of saving the capacity. There is a demand to view the moving image data from the PACS server.

According to the ultrasound diagnostic apparatuses described in JP 2013-312, JP 2017-182244, all of the image data is stored in one data storage server or image management apparatus and access is made to the above. The capacity of the one data storage server or the image management apparatus cannot be reduced.

The problem to be solved by the present embodiment corresponding to the above item is to easily access to the moving image data of the ultrasound image from the PACS server.

Here, in the ultrasound image management system 1, the patient and the person who performs the examination enter the first examination room E1 and performs the examination in which the scanning of the subject for the ultrasound image is performed using the ultrasound diagnostic apparatus 40.

In the ultrasound diagnostic apparatus 40 in the first examination room E1, for example, the person who performs the examination inputting the instruction to execute the moving image storage process through the operation inputter 416 functions as a trigger and the system controller 411 performs the moving image storage process according to the moving image storage program stored in the ROM.

The moving image management process shown in FIG. 11 includes a moving image storage step including the moving image storage process of the ultrasound diagnostic apparatus 40 and the moving image display step which displays the moving image data.

As the moving image storage step, in response to various input from the operator through the operation inputter 416, the system controller 411 of the ultrasound diagnostic apparatus 40 controls the transmitter 412, the receiver 413, and the probe switch 414 to transmit and receive the ultrasound from the ultrasound probe 42A, 42B, or 42C, scan the object, generate moving image data of the ultrasound image, display the moving image data on the display 417, and store the moving image data in the storage 418 (step S101). Then, through the communicator 419, the system controller 411 of the ultrasound diagnostic apparatus 40 accesses to the NAS 20, transmits the moving image data of the ultrasound image generated in step S101 to the NAS 20, and makes a request that the moving image data be stored (step S102).

The controller of the NAS 20 receives the moving image data of the ultrasound image transmitted in step S102 from the ultrasound diagnostic apparatus 40 through the communicator of the NAS 20 and stores the received moving image data of the ultrasound image in the storage of the NAS 20 (step S111).

Then, the system controller 411 of the ultrasound diagnostic apparatus 40 generates the image of the two-dimensional code including the moving image access information as the information for access by the PACS server 10, combines the image with the still image data of one frame in the moving image data of the ultrasound image stored in step S111, and generates the still image data of the ultrasound image including the image of the two-dimensional code (step S103). The moving image access information in step S103 is address information showing the storage position of the moving image data of the ultrasound image stored in the NAS 20 in step S111. The one frame in step S103 is the first frame of the moving image data in the ultrasound image, for example.

Then, the system controller 411 of the ultrasound diagnostic apparatus 40 accesses to the PACS server 10 through the communicator 419, transmits the still image data of the ultrasound image generated in step S103 to the PACS server 10 and requests the storage (step S104).

The controller of the PACS server 10 receives the still image data of the ultrasound image transmitted in step S104 from the ultrasound diagnostic apparatus 40 through the communicator of the PACS server 10 and stores the received still image data of the ultrasound image in the storage of the PACS server 10 (step S121).

Then, as the moving image display step, in response to the input of the viewing request of the moving image data of the ultrasound image from the person who performs the examination through the operator of the PACS viewer 30, first, the controller of the PACS viewer 30 transmits through the communicator of the PACS viewer 30 a request for the list of the still image data of the ultrasound image to the PACS server 10 (step S131).

Then, the controller of the PACS server 10 receives the request for the list of the still image data of the ultrasound image from the PACS viewer 30 through the communicator of the PACS server 10, and in response to the received request, the controller of the PACS server 10 reads all still image data of the ultrasound image from the storage of the PACS server 10 (step S122). Then, the controller of the PACS server 10 transmits the still image data of the ultrasound image read in step S122 to the PACS viewer 30 through the communicator of the PACS server 10 (step S123).

The controller of the PACS viewer 30 receives the still image data of the ultrasound image transmitted in step S123 from the PACS server 10 through the communicator of the PACS viewer 30. The controller of the PACS viewer 30 displays on the display the received still image data of the ultrasound image as a list. In response to an input of selection of the still image data of the ultrasound image as the target to be viewed from the person who performs the examination through the operator of the PACS viewer 30, the two-dimensional code included in the selected still image data of the ultrasound image is decoded and the moving image access information is obtained (step S132). Then, the controller of the PACS viewer 30 transmits to the NAS 20 the request for the moving image data corresponding to the selected still image data including the moving image access information obtained in step S132 through the communicator of the PACS viewer 30 (step S133).

The controller of the NAS 20 receives the request for the moving image data of the ultrasound image from the PACS viewer 30 through the communicator of the NAS 20, and in response to the received request, the controller of the NAS 20 reads the moving image data of the ultrasound image from the storage of the NAS 20 (step S112). The controller of the NAS 20 transmits the moving image data of the ultrasound image read in step S112 to the PACS viewer 30 through the communicator of the NAS 20 (step S113).

Then, the controller of the PACS 30 receives the moving image data of the ultrasound image transmitted in step S113 from the NAS 20 through the communicator of the PACS viewer 30 and displays on the display the received moving image data of the ultrasound image (step S134).

According to the moving image management process, by accessing to the still image data of the ultrasound image stored in the PACS server 10, the moving image access information of the still image data is obtained, and it is possible to easily access to the moving image data of the ultrasound image stored in the NAS 20. According to the above configuration, even the medical facilities that would like to store only the still image data in the PACS server 10 can centrally manage the moving image data of the ultrasound image with low costs by adding the NAS 20 which is cheap. The moving image access information (two-dimensional code) is combined with the still image data. Therefore, even if the PACS viewer 30 cannot obtain the moving image access information (decode the two-dimensional code), the moving image data of the ultrasound image can be viewed by installing additional software on the PC of the person who performs the examination (for example, if the click of the mouse of the PC is monitored, and there is a two-dimensional code in the position where the mouse is clicked, the moving image data of the ultrasound image of the moving image access information obtained by decoding the two-dimensional code can be played and viewed). Moreover, even if the PC is not used, the moving image access information (two-dimensional code) displayed on the PACS viewer 30 can be captured using the portable terminal 50A of the person who performs the examination. With this, the moving image data of the ultrasound image which can be obtained from the moving image access information can be played and viewed, and this is convenient.

### 1-2-5. Ultrasound Diagnostic Gel Container

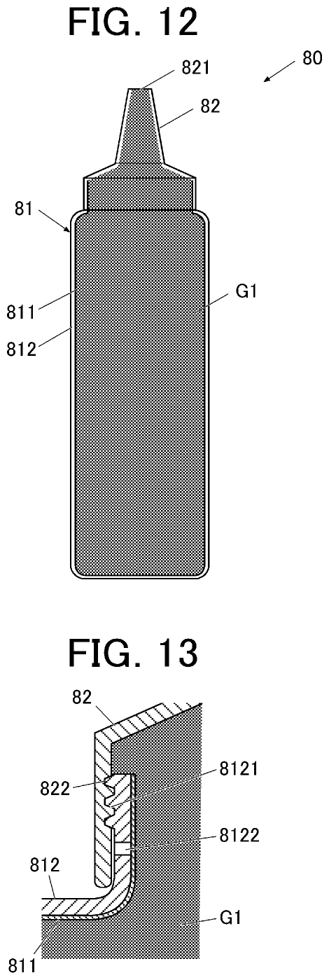

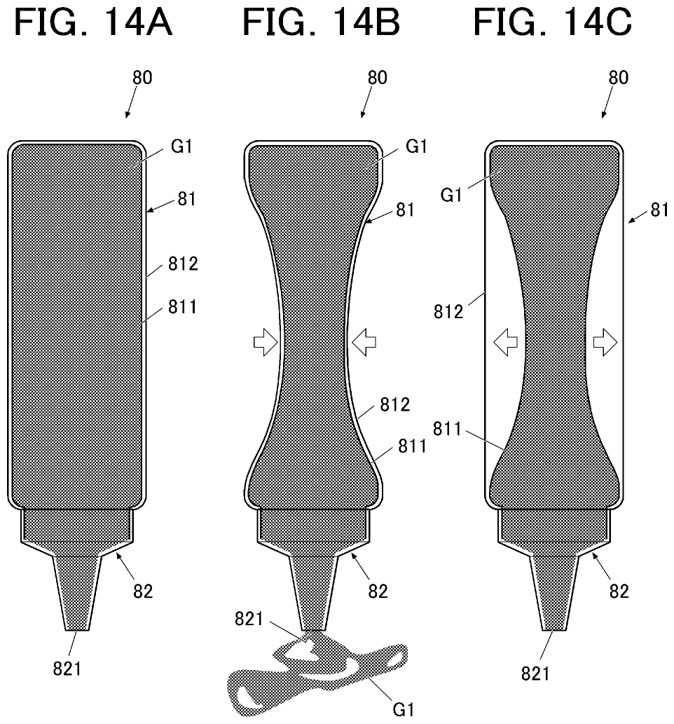

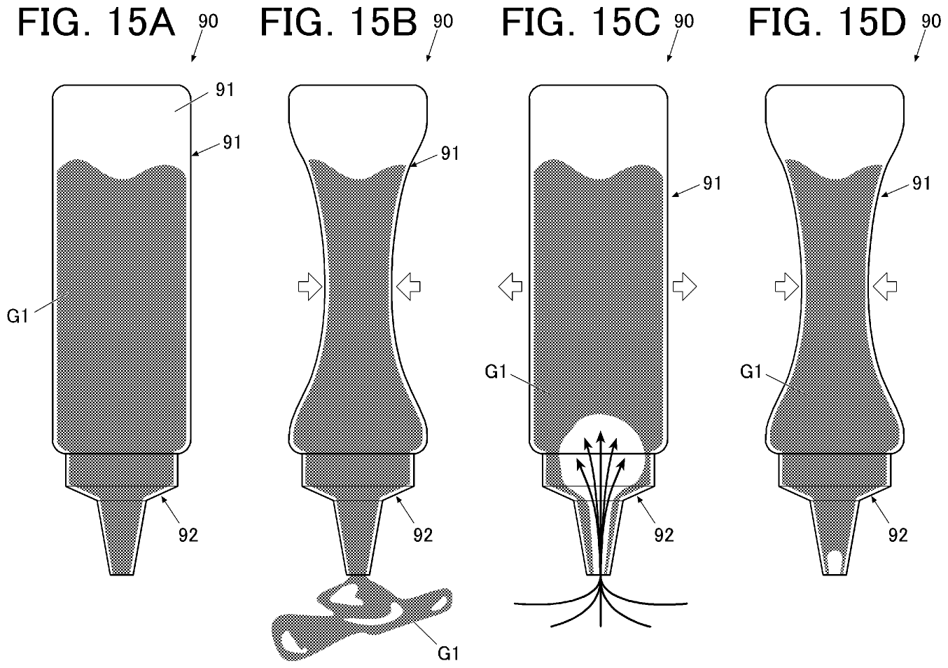

With reference to FIG. 12 to FIG. 15B, the apparatus configuration of the ultrasound diagnostic gel container 80 used in the ultrasound diagnostic apparatus 40 and the operation in use are described. FIG. 12 shows an external appearance of the ultrasound diagnostic gel container 80. FIG. 13 is a partial cross-section diagram showing the ultrasound diagnostic gel container 80. FIG. 14A is a schematic diagram showing the ultrasound diagnostic gel container 80 in a first stage of use. FIG. 14B is a schematic diagram showing the ultrasound diagnostic gel container 80 in a second stage of use. FIG. 14C is a schematic diagram showing the ultrasound diagnostic gel container 80 in a third stage of use. FIG. 15A is a schematic diagram showing the ultrasound diagnostic gel container 90 in a first stage of use. FIG. 15B is a schematic diagram showing the ultrasound diagnostic gel container 90 in a second stage of use. FIG. 15C is a schematic diagram showing the ultrasound diagnostic gel container 90 in a third stage of use. FIG. 15D is a schematic diagram showing the ultrasound diagnostic gel container 90 in a fourth stage of use.

As shown in FIG. 6, when the scanning for the ultrasound image is performed, the ultrasound diagnostic gel is applied to an ultrasound transmission/reception surface at a tip of the ultrasound probe (acoustic lens). For example, the ultrasound diagnostic gel is a sterilized gel including water as the main component, specifically, a mixture of carboxyvinyl polymer and water. The ultrasound diagnostic gel includes acoustic impedance between the acoustic impedance (of the acoustic lens) of the ultrasound probe and the acoustic impedance of the subject, and is used to prevent the ultrasound from being reflected between the above.

As the ultrasound diagnostic gel container, for example, there is a well-known ultrasound diagnostic container including the ultrasound diagnostic gel container main body and a main body cap attached to the container main body and including a spout (JP 2004-189335). The person who performs the examination presses the container main body with his hand to discharge the ultrasound diagnostic gel. There is also a well-known lubricant supplying container which includes a bottle for the ultrasound diagnostic gel (lubricant), a pump dispenser, and a foot switch. The person who performs the examination presses a foot switch to discharge the ultrasound diagnostic gel (utility model JP 3143923).

A conventional hand-held ultrasound diagnostic gel container 90 which is similar to the ultrasound diagnostic gel container described in JP 2004-189335 and which is easy to handle with a simple configuration is considered. As shown in FIG. 15A, the ultrasound diagnostic gel container 90 includes a container main body 91 with a one layer structure in which the ultrasound diagnostic gel G1 is stored and a main body lid 92 including a discharge opening.

When the ultrasound diagnostic gel G1 is discharged from the ultrasound diagnostic gel container 90, as shown in FIG. 15A, as the first stage of use, the person who performs the examination holds the ultrasound diagnostic gel container 90 upside down in his hand and moves the ultrasound diagnostic gel G1 to the discharge opening side of the main body lid 92. If this move is not sufficient, the person who performs the examination holds the ultrasound diagnostic gel container 90 upside down with his hand and shakes the ultrasound diagnostic gel container 90 up and down to guide the ultrasound diagnostic gel G1 to move downward. As shown in FIG. 15A, in order to make the ultrasound diagnostic gel G1 easy to see, the container main body 91 and the main body lid 92 are shown transparent, and this can be similarly said for FIG. 15B to FIG. 15D, and FIG. 14A to FIG. 14C (container main body 81 and main body lid 82).

Then, as shown in FIG. 15B, as the second stage of use, when the person who performs the examination grips the container main body 91 with the hand again and applies pressure from outside, the ultrasound diagnostic gel G1 is discharged from the discharge opening of the main body lid 92. As shown in FIG. 15C, as the third stage of use, when the person who performs the examination releases the grip on the container main body 91 with the hand, the air flows backward from the discharge opening of the main body lid 92 into the container main body 91.

Then, as shown in FIG. 15D, as the fourth stage of use, even if the person who performs the examination grips the container main body 91 with the hand and applies pressure from outside, the air which flowed backward in the container main body 91 is emitted and the ultrasound diagnostic gel G1 is not discharged. In this case, while in the third stage, the person who performs the examination needs to hold the ultrasound diagnostic gel container 90 upside down in his hand and shake the ultrasound diagnostic gel container 90 up and down to move the ultrasound diagnostic gel G1 downwards and this is a heavy burden on the person who performs the examination

The problem to be solved by the present embodiment corresponding to the above item is to easily discharge the ultrasound diagnostic gel with the hand and to reduce the burden of the person who performs the examination.

As shown in FIG. 12, the ultrasound diagnostic gel container 80 is used in the ultrasound probes 42A, 42B, and 42C of the ultrasound diagnostic apparatus 40 of the present embodiment. The ultrasound diagnostic gel container 80 includes a container main body 81 and a main body lid 82. The container main body 81 includes an inner side container 811 and an outer side container 812. The container main body 81 is a delaminating bottle which is open on the upper side, and which includes a two layer configuration in which the inner side container 811 is layered in the inner side of the outer side container 812 in a separable manner.

The outer side container 812 includes polyethylene resin and polypropylene resin and is positioned to cover the outer side of the inner side container 811. The inner side container 811 includes polyimide type synthetic resin and ethylene vinyl alcohol copolymer resin which are not compatible with the resin forming the outer side container 812. The ultrasound diagnostic gel G1 is stored in the inner side container 811.

The main body lid 82 is a lid including the polyethylene resin and including an opening portion on the bottom surface where the opening portion of the container main body 81 is attached. The main body lid 82 includes a discharge opening 821 of the ultrasound diagnostic gel G1 at the tip. The main body lid 82 includes a shape tapered in two steps diagonally downward so as to spread outside from the discharge opening 821.

With reference to FIG. 13, the attaching portion of the main body lid 82 to the container main body 81 is described. In the opening portion of the container main body 81, the outer side container 812 is layered on the outer side of the inner side container 811. A male screw 8121 is provided on the upper side of the inner side container 811, and an intake opening 8122 is provided below the male screw 8121. The main body lid 82 includes the female screw 822 in the position of the opening portion corresponding to the male screw 8121. The female screw 822 fits with the male screw 8121 to attach the main body lid 82 to the container main body 81. In this state, the ultrasound diagnostic gel G1 is filled in the inner side container 811 and the main body lid 82. When the ultrasound diagnostic gel container 80 is not used, at least an over cap (not shown) which covers the discharge opening 821 can be attached to the main body lid 82.

Next, with reference to FIG. 14A to FIG. 14C, the operation to discharge the ultrasound diagnostic gel G using the ultrasound diagnostic gel container 80 is described. When the ultrasound diagnostic gel G1 is discharged from the ultrasound diagnostic gel container 80, first, as shown in FIG. 14A, as the first stage of use, the person who performs the examination holds the ultrasound diagnostic gel container 80 in his hand upside down. Since the ultrasound diagnostic gel G1 is filled in the inner side container 811, the person who performs the examination does not have to hold the ultrasound diagnostic gel container 80 upside down in his hand and shake the ultrasound diagnostic gel container 80 up and down.

As shown in FIG. 14B, as the second stage of use, when the person who performs the examination grips the container main body 81 from outside with his hands and applies pressure, the ultrasound diagnostic gel G1 is discharged from the discharge opening 821 of the main body lid 82. Then, as shown in FIG. 14C, as the third stage of use, when the person who performs the examination releases the grip by the hand on the container main body 81, the outer side container 812 returns to the original state, but at the same time the air from outside flows between the outer side container 812 and the inner side container 811 through the intake opening 8122. With this, the shape of the inner side container 811 is maintained as is.

Therefore, since the ultrasound diagnostic gel G1 is filled in the inner side container 811 without the air flowing backward in the ultrasound diagnostic gel container 80, there is no need for the person who performs the examination to hold the ultrasound diagnostic gel container 80 in his hand upside down and shake the ultrasound diagnostic gel container 80 up and down when the ultrasound diagnostic gel container 80 is used next time.

As described above, according to the ultrasound diagnostic gel container 80, the container main body 81 includes a two layer structure including the inner side container 811 and the outer side container 812. Therefore, the ultrasound diagnostic gel G1 can be easily discharged with the hand Even if the ultrasound diagnostic gel G1 is discharged, the air flowing backwards from the discharge opening 821 is prevented. Therefore there is no need for the person who performs the examination to shake the ultrasound diagnostic gel container 80, and the burden of the person who performs the examination can be reduced.

### 2. First Modification

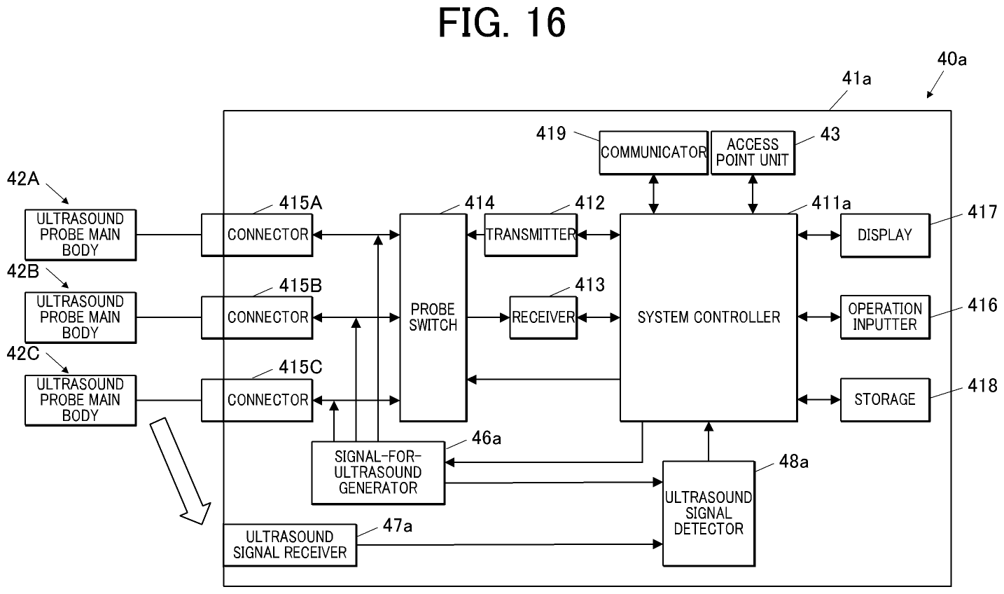

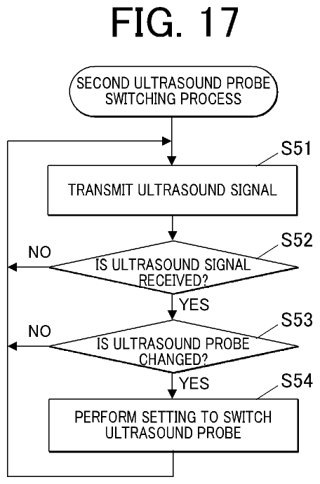

A first modification of the above embodiment is described with reference to FIG. 16 and FIG. 17. FIG. 16 is a block diagram showing a functional configuration of the ultrasound diagnostic apparatus 40a. FIG. 17 is a flowchart showing the second ultrasound probe switching process.

### 2-1. Apparatus Configuration

According to the modification, the portions different from the above embodiment are described and the description of the similar portions are emitted. In this modification, instead of the ultrasound diagnostic apparatus 40 in the ultrasound image management system of the above embodiment, the ultrasound diagnostic apparatus 40a is used. In the ultrasound diagnostic apparatus 40, the ultrasound probes 42A, 42B, and 42C are imaged and the image is analyzed. In response to the analysis result, the used ultrasound probe is switched. According to the ultrasound diagnostic apparatus 40a, the ultrasound transmitted from the ultrasound probe which is used is received and the ultrasound probe is detected. With this, the ultrasound probe which is used is switched.

As shown in FIG. 16, the ultrasound diagnostic apparatus 40a includes an ultrasound diagnostic main body 41a and ultrasound probes 42A, 42B, and 42C. According to the ultrasound diagnostic main body 41a, the system controller 411 of the ultrasound diagnostic apparatus main body 41 (FIG. 3) of the present embodiment is changed to a system controller 411a, the imager 44 and the recognizer 45 are removed, and a signal-for-ultrasound generator 46a, an ultrasound signal receiver 47a, and an ultrasound signal detector 48a as a detector are added.

The system controller 411a is similar to the system controller 411. Instead of the first ultrasound probe switching program in the ROM, a second ultrasound probe switching program to perform the later-described second ultrasound probe switching process is stored.

The signal-for-ultrasound generator 46a includes a signal generator, and according to the control of the system controller 411a, a signal for ultrasound as a driving signal to transmit the ultrasound signal as the ultrasound for detecting the ultrasound probe is generated, and the signal is output to the connectors 415A, 415B, and 415C and the ultrasound signal detector. The signals for ultrasound input in the connectors 415A, 415B, and 415C are output to the ultrasound probes 42A, 42B, and 42C. The signals for ultrasound corresponding to the ultrasound probes 42A, 42B, and 42C are generated repeatedly in order in a loop, and the timing of output is different. The signal-for-ultrasound generator 46a outputs to the ultrasound signal detector 48a the ultrasound probe information showing to which of the ultrasound probes 42A, 42B, and 42C (connectors 415A, 415B, and 415C) the signal for ultrasound is output.

The ultrasound probes 42A, 42B, and 42C transmit the ultrasound signal in response to the signal for ultrasound corresponding to the ultrasound probes 42A, 42B, and 42C.

The ultrasound signal receiver 47a includes an oscillator (piezoelectric element). According to the control by the system controller 411a, the ultrasound signal transmitted from the ultrasound probe 42A, 42B or 42C is received, and the ultrasound received signal as the electric signal is generated. The ultrasound signal receiver 47a generates the ultrasound received signal of the ultrasound probe 42A, 42B or 42C in which the ultrasound transmission/reception surface faces nearby.

According to the control by the system controller 411a, in response to the ultrasound probe information input from the signal-for-ultrasound generator 46a and the ultrasound received signal input from the ultrasound signal receiver 47a, the ultrasound signal detector 48a generates the identification information for the ultrasound probe in which the ultrasound transmission/reception surface faces nearby as detection information and outputs the information to the system controller 411a.

### 2-2. Operation of Ultrasound Diagnostic Apparatus 40a

With reference to FIG. 17, the second ultrasound probe switching process performed by the ultrasound diagnostic apparatus 40a is described. For example, in the first examination room E1, the ultrasound diagnostic apparatus 40a is used to perform an examination on a plurality of patients in order. The person who performs the examination and the patient enter the first examination room E1 and the examination is started. The ultrasound probes 42A, 42B, and 42C are connected to the ultrasound diagnostic apparatus main body 41a of the ultrasound apparatus 40a in the first examination room E1.

In the examination, the person who performs the examination operates the ultrasound diagnostic apparatus 40a and the scanning of the subject for the ultrasound image is performed. When the ultrasound probe which is used is changed during the examination, the person who performs the examination holds the changed ultrasound probe and places the ultrasound probe toward the ultrasound signal receiver 47a (faces the ultrasound transmission/reception surface nearby).

For example, in the ultrasound diagnostic apparatus 40a, the input of the instruction to perform the second ultrasound probe switching process from the person who performs the examination through the operation inputter 416 functions as the trigger and the system controller 411a performs the second ultrasound probe switching process according to the second ultrasound probe switching program stored in the ROM.

As shown in FIG. 17, first, the system controller 411a uses the signal-for-ultrasound generator 46a to generate the signal for ultrasound, outputs the signal to the ultrasound probes 42A, 42B, and 42C, and transmits the ultrasound signal from the ultrasound probes 42A, 42B, and 42C (step S51). The transmission of the ultrasound signal for step S51 is performed at a timing which does not interfere with the transmission and reception of the ultrasound for the examination.

Then, the system controller 411a uses the ultrasound signal receiver 47a and the ultrasound signal detector 48a to determine whether the ultrasound signal of the ultrasound probe 42A, 42B or 42C is received according to the obtained detection information (step S52). When the ultrasound signal is not received (step S52; NO), the process progresses to step S51.

When the ultrasound signal is received (step S52; YES), the system controller 411a determines whether the ultrasound probe corresponding to the received ultrasound signal is not the ultrasound probe being used and the ultrasound probe is to be changed (step S53). When the ultrasound probe is not changed (step S53; NO), the process progresses to step S51.

When the ultrasound probe is changed (step S53; YES), the system controller 411 performs a switching setting in the probe switch 414 to electrically connect the path corresponding to the changed ultrasound probe corresponding to the received ultrasound signal as the path for the driving signal and the received signal (step S54). Then the process progresses to step S51.

According to the modification, the ultrasound diagnostic apparatus 40a is an ultrasound diagnostic apparatus to which the ultrasound probes 42A, 42B, and 42C are connected, and in which the ultrasound is transmitted and received using one of the ultrasound probes 42A, 42B, and 42C and the ultrasound image data is generated. The ultrasound diagnostic apparatus 40a includes the signal-for-ultrasound generator 46a which generates the signal for ultrasound and outputs the signal for ultrasound to the ultrasound probes 42A, 42B, and 42C in order, the ultrasound signal receiver 47a which receives the ultrasound signal transmitted from the ultrasound probe according to the signal for ultrasound, the ultrasound signal detector 48a which detects the ultrasound probe from which the ultrasound signal is received, and the system controller 411a which switches and sets the detected ultrasound probe to be the ultrasound probe to be used. Therefore, by using the existing ultrasound probe, without adding anything to the ultrasound probe, the ultrasound probe to be used can be easily switched by simply placing the ultrasound transmission/reception surface of the ultrasound probe to be used toward the ultrasound signal receiver 47a of the ultrasound diagnostic apparatus 40a.

### 3. Second Modification

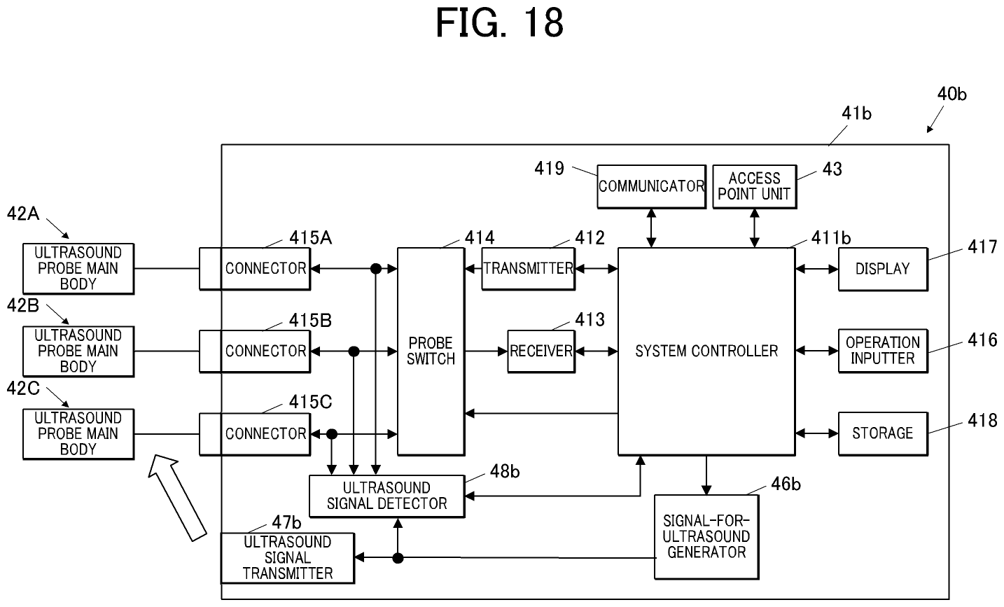

The second modification according to the present embodiment is described with reference to FIG. 18. FIG. 18 is a block diagram showing the functional configuration of the ultrasound diagnostic apparatus 40b.

### 3-1. Apparatus Configuration

According to the modification, the portions different from the above-described embodiment are mainly described and the description of the similar portions is omitted. Instead of the ultrasound diagnostic apparatus 40 in the ultrasound image management system 1 according to the above embodiment, the ultrasound diagnostic apparatus 40b is used in the present modification. According to the ultrasound diagnostic apparatus 40b, by detecting the ultrasound received signal received by the ultrasound probe which is used, the ultrasound probe which is used is switched.

As shown in FIG. 18, the ultrasound diagnostic apparatus 40b includes the ultrasound diagnostic apparatus main body 41b and the ultrasound probes 42A, 42B, and 42C. According to the ultrasound diagnostic apparatus 41b, the system controller 411 of the ultrasound diagnostic apparatus main body 41 (FIG. 3) according to the above embodiment is changed to the system controller 411b, the imager 44 and the recognizer 45 are removed, and the signal-for-ultrasound generator 46b, the ultrasound signal transmitter 47b, and the ultrasound detector 48b as the detector are added.

The system controller 411b is similar to the system controller 411, and instead of the first ultrasound probe switching program in the ROM, the second ultrasound probe switching program to perform the later-described second ultrasound probe switching process is stored.

The signal-for-ultrasound generator 46b includes a signal generator. According to the control by the system controller 411b, the signal-for-ultrasound generator 46b generates the signal for ultrasound as the driving signal to transmit the ultrasound signal as the ultrasound for detecting the ultrasound probe and outputs the signal for ultrasound to the ultrasound signal transmitter 47b and ultrasound signal detector 48b. The generated signals for ultrasound are the same signals regardless of the ultrasound probes 42A, 42B, and 42C.

The ultrasound signal transmitter 47b includes an oscillator (piezoelectric element) and according to the control by the system controller 411b, the ultrasound signal transmitter 47b generates the ultrasound signal according to the signal for ultrasound input from the signal-for-ultrasound generator 46b and transmits the ultrasound signal.

The ultrasound probes 42A, 42B, and 42C can receive the ultrasound signal transmitted from the ultrasound signal transmitter 47b.

According to the control by the system controller 411b, the ultrasound signal detector 48b detects the ultrasound received signal as the electric signal input through the connectors 415A, 415B, and 415C from the ultrasound probes 42A, 42B, and 42C. The ultrasound signal detector 48b outputs to the system controller 411b the identification information of the ultrasound probe corresponding to the detected ultrasound received signal as the detection information.

### 3-2. Operation of Ultrasound Diagnostic Apparatus 40b

The second ultrasound probe switching process performed by the ultrasound diagnostic apparatus 40b is described. The second ultrasound probe switching process is similar to the second ultrasound probe switching process of the first modification (FIG. 17) and the different portions are mainly described.

In step S51 of the second ultrasound probe switching process, the system controller 411b controls the signal-for-ultrasound generator 46b to generate the signal for ultrasound. The signal for ultrasound is output to the ultrasound signal transmitter 47b and the ultrasound signal is transmitted.

In step S52, the system controller 411b uses the ultrasound signal transmitter 47b and the ultrasound signal detector 48 to determine whether the ultrasound probes 42A, 42B, or 42C received the ultrasound signal according to the obtained detection information. In step S53, the system controller 411b determines whether the ultrasound probe which is used is changed according to the detection information obtained in step S52.

According to the above modification, the ultrasound diagnostic apparatus 40b is an ultrasound diagnostic apparatus to which the ultrasound probes 42A, 42B, and 42C are connected and in which one of the ultrasound probes 42A, 42B, and 42C is used to transmit and receive the ultrasound to generate the ultrasound image data. The ultrasound diagnostic apparatus 40b includes the signal-for-ultrasound generator 46b which generates the signal for ultrasound, the ultrasound signal transmitter 47b which transmits the ultrasound signal according to the signal for ultrasound, the ultrasound signal detector 48b which detects which ultrasound probe received the ultrasound signal, and the system controller 411a which switches and sets the detected ultrasound probe to be the ultrasound probe which is used. Therefore, by using the existing ultrasound probe, without adding anything to the ultrasound probe, the ultrasound probe to be used can be easily switched by simply placing the ultrasound transmission/reception surface of the ultrasound probe desired to be used toward the ultrasound signal transmitter 47b of the ultrasound diagnostic apparatus 40a.

The description of the embodiments and the modifications merely describe examples of the ultrasound diagnostic apparatus, the ultrasound probe switching method, and the storage medium suitable for the present invention, and the present invention is not limited to the above.

The detailed configuration and the detailed operation included in the ultrasound image management system 1 as described in the embodiments and modifications can be changed without leaving the scope of the present invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasound diagnostic apparatus to which a plurality of ultrasound probes are connected and which transmits and receives ultrasound using one of the plurality of ultrasound probes to generate ultrasound image data, the ultrasound diagnostic apparatus comprising:
a detector which optically detects the ultrasound probe which is used among the plurality of ultrasound probes; and
a hardware processor which switches and sets the detected ultrasound probe to be the ultrasound probe which is used.

2. The ultrasound diagnostic apparatus according to claim 1, wherein the detector images an identifier including identification information of the ultrasound probe attached to the ultrasound probe and detects the identification information of the ultrasound probe included in the identifier.

3. The ultrasound diagnostic apparatus according to claim 1, wherein the detector images the ultrasound probe and detects the ultrasound probe from an external shape of the ultrasound probe.

4. The ultrasound diagnostic apparatus according to claim 1, wherein,
the detector images an identifier including contents of a setting for an examination using the ultrasound probe attached to the ultrasound probe and detects the contents of the setting for the examination included in the identifier, and
the hardware processor sets the detected contents of the setting for the examination.

5. The ultrasound diagnostic apparatus according to claim 1, wherein,
the detector images the ultrasound probe and detects predetermined movement of the ultrasound probe; and
the hardware processor performs a command corresponding to the detected predetermined movement.

6. An ultrasound diagnostic apparatus to which a plurality of ultrasound probes are connected and which transmits and receives ultrasound using one of the plurality of ultrasound probes to generate ultrasound image data, the ultrasound diagnostic apparatus comprising:
a signal-for-ultrasound generator which generates a signal for ultrasound and outputs the signal for ultrasound to the plurality of ultrasound probes in order;
an ultrasound signal receiver which receives an ultrasound signal transmitted from the ultrasound probe in response to the signal for ultrasound;
a detector which detects the ultrasound probe from which the ultrasound signal is received; and
a hardware processor which switches and sets the detected ultrasound probe to be the ultrasound probe which is used.

7. An ultrasound diagnostic apparatus to which a plurality of ultrasound probes are connected and which transmits and receives ultrasound using one of the plurality of ultrasound probes to generate ultrasound image data, the ultrasound diagnostic apparatus comprising:
a signal-for-ultrasound generator which generates a signal for ultrasound;
an ultrasound signal transmitter which transmits an ultrasound signal in response to the signal for ultrasound;
a detector which detects the ultrasound probe which received the ultrasound signal; and
a hardware processor which switches and sets the detected ultrasound probe to be the ultrasound probe which is used.

8. An ultrasound probe switching method used in an ultrasound diagnostic apparatus to which a plurality of ultrasound probes are connected and which transmits and receives ultrasound using one of the plurality of ultrasound probes to generate ultrasound image data, the ultrasound probe switching method comprising:
optically detecting the ultrasound probe which is used among the plurality of ultrasound probes; and
switching and setting the detected ultrasound probe to be the ultrasound probe which is used.

9. A non-transitory computer-readable storage medium storing a program causing a computer in an ultrasound diagnostic apparatus to which a plurality of ultrasound probes are connected and which transmits and receives ultrasound using one of the plurality of ultrasound probes to generate ultrasound image data, the program comprising:
optically detecting the ultrasound probe which is used among the plurality of ultrasound probes; and
switching and setting the detected ultrasound probe to be the ultrasound probe which is used.
